(19) United States
(12) Patent Application Publication
(10) Pub. No.: US 2024/0346906 A1
(43) Pub. Date: Oct. 17, 2024

(54) MOUNTABLE APPARATUS FOR PROVIDING USER DATA MONITORING AND COMMUNICATION IN HAZARDOUS ENVIRONMENTS

(71) Applicant: AI TECH HOLDINGS, INC., Chicago, IL (US)

(72) Inventors: Alexander Gorsuch, Chicago, IL (US); Paul Couston, Chicago, IL (US); Thomas Kaufmann, Chicago, IL (US); Molly Izzi, Chicago, IL (US); Dominic Zerillo, Chicago, IL (US)

(21) Appl. No.: 18/291,194

(22) PCT Filed: Jul. 26, 2022
(86) PCT No.: PCT/US22/38393

§ 371 Date: Jan. 22, 2024

Related U.S. Application Data
(60) Provisional application No. 63/226,725, filed on Jul. 28, 2021.
(60) Provisional application No. 63/333,805, filed on Apr. 22, 2022.
(60) Provisional application No. 63/311,290, filed on Feb. 17, 2022.

(51) Int. Cl. G08B 21/04; A61B 5/00; A62B 18/08; G01C 21/16

(52) U.S. Cl. CPC G08B 21/0446; A61B 5/6803; G01C 21/1652; G01C 21/1656; A62B 18/08

(57) ABSTRACT

A system is disclosed comprising: (a) apparatus for mounting on personal protective equipment (PPE) of a user located on premises in a hazardous environment. The apparatus is configured for providing data monitoring and communication of the user for remote review, analyses and/or user deployment and navigation guidance in hazardous environments. The apparatus includes a user tracking device for tracking location of the user on premises comprising: an inertial measurement unit for measuring and reporting acceleration, velocity and position data of the user on premises; an infrared camera for creating image data as the user moves through the premises; a GPS receiver; an ultrasound sensor; and a microcontroller unit for processing and transmitting data from the inertial measurement unit, ultrasound sensor and GPS remotely; and (b) a mounting accessory for mounting the user tracking device to the user's personal protective equipment.

### CROSS-REFERENCE TO RELATED APPLICATIONS

This application claims priority to U.S. provisional application No. 62/226,725, filed Jul. 28, 2021 entitled “Mountable Sensor Modules For Protective Equipment”, U.S. provisional application No. 63/333,805, filed Apr. 22, 2022, entitled “Location Tracking System of Users in Hazardous Environments” and U.S. provisional application No. 63/311,290, filed Feb. 17, 2022, entitled “Apparatus For Hands Free Communication and Biometric Monitoring in Hazardous Environments”, which are all incorporated by reference herein.

### FIELD OF THE INVENTION

The present invention relates to a mountable apparatus for providing user data monitoring and communication.

### BACKGROUND OF THE INVENTION

Personal protective equipment (PPE), such as masks, helmets, gloves, and body armor are worn by operators in austere environments. This PPE is often paired with other PPE such as fire-proof hoods, air tanks and hoses, boots, and protective suits. Together, these pieces of equipment allow for reliable respiration, fire and water resistance, protection from hazardous gas and other aspects of user protection. These PPE systems are used in many industries, such as fire service, industrial work, hazardous materials or gases manufacturing, mining and raw materials processing, as well as avionic and marine/nautical mechanics, among others.

Current PPE solutions accomplish the base function of protection. However, due to the nature of protection, the equipment can decrease peripheral vision, make it difficult to communicate, and/or severely limit aspects of human sensor perception.

### SUMMARY OF THE INVENTION

A mountable apparatus for providing user data monitoring and communication in hazardous environments.

In accordance with an embodiment of the present disclosure, an apparatus for mounting on personal protective equipment (PPE) of a user located on premises in a hazardous environment, the apparatus configured for providing data monitoring and communication of the user for remote review, analyses and/or user deployment and navigation guidance in the hazardous environments to enhance incident command capability, the personal protective equipment including a helmet and/or a mask, the apparatus comprising: (a) one or more modules including: an inertial measurement unit for measuring and reporting acceleration, velocity and position data of the user on premises; an infrared camera for creating image data as the user moves through the premises; a GPS receiver for generating geolocation data of the user via satellite imagery as the user enters the premises; an ultrasound sensor for generating data relating to the distance between the user and objects on premises; and a microcontroller unit for processing and transmitting data from the inertial measurement unit, infrared camera, ultrasound sensor and GPS to a remote computer system; and (b) a mounting accessory for mounting the one or more modules to the user's personal protective equipment.

In accordance with yet another embodiment of the present disclosure, a system comprising: (a) apparatus for mounting on personal protective equipment (PPE) of a user located on premises in a hazardous environment, the apparatus configured for providing data monitoring and communication of the user for remote review, analyses and/or user deployment and navigation guidance in the hazardous environments to enhance incident command capability, the personal protective equipment including a helmet and/or a mask, the apparatus including a user tracking device for tracking location of the user on premises comprising: an inertial measurement unit for measuring and reporting acceleration, velocity and position data of the user on premises; an infrared camera for creating image data as the user moves through the premises; a GPS receiver for generating geolocation data of the user via satellite imagery as the user enters the premises; an ultrasound sensor for generating data relating to the distance between the user and objects on premises; and a microcontroller unit for processing and transmitting data from the inertial measurement unit, ultrasound sensor and GPS remotely; and (b) a mounting accessory for mounting the user tracking device to the user's personal protective equipment.

In accordance with another embodiment of the present disclosure, an apparatus that is configured as one or more modules or components to be mounted on a user on premises in hazardous environments, the apparatus comprising: (a) a first user tracking device for tracking location of a user on the premise, the first user tracking device including: an inertial measurement unit for measuring and reporting acceleration, velocity and position data of the user on premises; a GPS receiver for generating geolocation data of the user via satellite imagery as the user enters the premises; and a microcontroller unit for processing and transmitting data from the inertial measurement unit and GPS to a remote computer system; and (b) a second user tracking device including: an inertial measurement unit for measuring and reporting acceleration, velocity and position data of the user on premises; a GPS receiver for generating geolocation data of the user via satellite imagery as the user enters the premises; and a microcontroller unit for processing and transmitting data from the inertial measurement unit and GPS to the microcontroller the remote computer system, wherein the first tracking device and second tracking device are configured to transmit data therebetween; and (c) a first mounting accessory and second mounting accessory for mounting the first user tracking device and the second user tracking device respectively to the user.

### BRIEF DESCRIPTION OF DRAWINGS

FIG. 1 depicts a diagram of an environment in which an example location tracking system of users in hazardous environments operates.

FIG. 2 depicts a diagram of the example location tracking system in FIG. 1.

FIG. 3 depicts a flow diagram of the platform steps for performing the function of the example tracking system of FIG. 2.

FIGS. 4A and 4B depict front and side views of an example mounting accessory and module in an exploded configuration.

FIG. 4C depicts the module and mounting assembly in FIGS. 4A and 4B in an assembled configuration.

FIG. 5A depicts a front view of another mounting accessory and module as installed within the mounting accessory.

FIG. 5B depicts a side exploded view of another example mounting accessory and module in FIG. 5A.

FIGS. 6A and 6B depict side views of another example mounting accessory and sensor module.

FIG. 6C is a front view of the mounting accessory and module in FIGS. 6A and 6B.

FIG. 7 depicts an inside view of the module shown in FIGS. 6A-6C.

FIGS. 8A and 8B depict front views of another example mounting accessory and modules in post and pre installment configurations.

FIG. 8C depicts a side view of the module depicted in FIGS. 8A and 8B.

FIGS. 8D and 8E depict enlarged views of the clamp or clamping mechanism onto the arms of the mounting accessory depicted in FIGS. 8A and 8B.

FIG. 8F depicts a front view of the mount device in FIGS. 8A and 8B in pre-deployed mounting to a mask.

FIG. 9 depicts a view of another example mounting accessory.

FIG. 10 depicts a front view of another example mounting accessory for a module.

FIGS. 11A-11B depict front views of another example mounting accessory and module in pre and post installed configurations.

FIGS. 12A-12B depict views of an example of a user helmet along with the module depicted in FIGS. 6A-6C.

FIG. 13A depicts a front sectional view of another example mounting accessory that is used for mounting a module to a user's/wearer's mask.

FIG. 13B depicts an exploded sectional view of the mounting accessory, module and mask depicted in FIG. 13A.

FIG. 13C depicts a side perspective view of mounting accessory depicted in FIG. 13A.

FIG. 14A depicts a front perspective view of another example mounting accessory with dual modules mounted to a helmet along its brim.

FIG. 14B depicts a sectional view of the rear of the helmet shown in FIG. 14A.

FIG. 14C depicts a sectional view of the side of the helmet shown in FIG. 14A.

FIGS. 15A-15B depict front views of another example of mounting accessories for mounting modules to a SCBA mask.

FIGS. 16A-16C depict various views of another example of a combined mounting accessory and module that functions to clamp the module to a helmet.

FIG. 16D depicts an exploded view of the combined mounting accessory and module in FIGS. 16A-16C.

FIG. 17A depicts a side view of a module mounted to a user's foot.

FIG. 17B depicts a perspective view of the module in FIG. 17A.

FIG. 17C depicts a perspective view of the module in FIG. 17A with the housing or enclosure open exposing an IC board.

FIG. 18A depicts a side rear view of another module mounted to a wearer or user along his/her ankle using a clip as a mounting accessory.

FIG. 18B depicts a rear perspective view of the module in FIG. 18A.

FIG. 18C depicts a front perspective view of the module in FIG. 18A with its door open exposing internal components.

FIG. 18D depicts a side view of the clip attached to the inside of the module in FIG. 18A.

FIG. 19 depicts a block diagram of data flow of the system shown in FIG. 2.

### DETAILED DESCRIPTION OF THE INVENTION

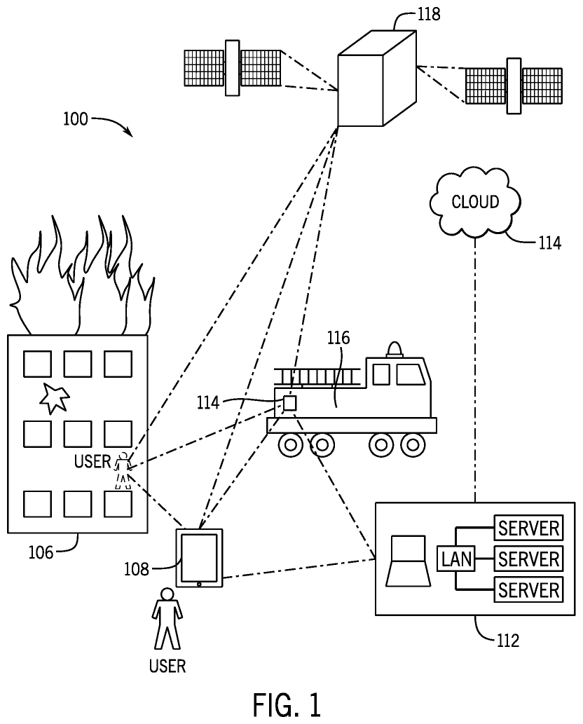

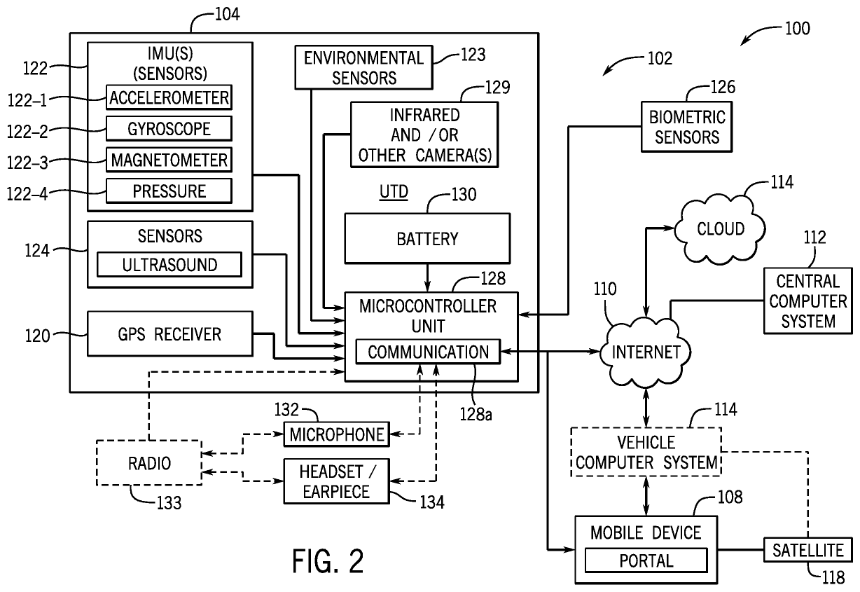

FIG. 1 depicts a diagram of an environment or system in which an example location tracking (localization) system 100 of users in hazardous environments operates. FIG. 2 depicts a diagram of the location tracking system in FIG. 1. Specifically, system 100 is configured to function in such hazardous environments including severe and challenging austere environments. Examples of such austere environments include fires in residential, industrial, commercial, or other installations and accompanying fumes, toxic gas release and exposure and/or other harmful conditions. Additional examples of other environments include non-fire related environments such as military and law enforcement conducted operations, hazardous materials and confined space entry.

System 100 includes apparatus 102 that is configured to be mounted on a user without compromising the user's equipment or changing the way in which the user accomplishes the task at hand. The mounting may be on the user's skin, clothing etc. or on items of a user's personal protective equipment (PPE). PPE as known to those skilled in the art is worn by the user to minimize exposure to hazards that cause injuries and illnesses. These injuries and illnesses may result from contact with chemical, radiological, physical, electric, mechanical or other workplace hazards. PPE may include items such as gloves, safety glasses, shoes, earplugs or muffs, hard hats, respirator, coveralls, vests and full body suits.

Apparatus 102 is configured as one or more hands free modules or component(s) that provide user data monitoring and/or communication for remote review, analyses and user guidance in hazardous environments. The user data monitoring and/or communication includes, for example, voice communication, biometric monitoring, environmental monitoring, image visualization, user location tracking and/or other functions of a user as described below in detail. The data collected will also be used to improve remote incident command capability. This will help incident command to (1) gain insight into a user's health status, PPE status as well an internal building structure and to (2) guide user (firefighter) deployment and navigation as described hereinbelow.

The modules are configured to be mounted on user PPE or directly on the user (wearer). (Modules as described herein may also be referred to as sensor modules.)

Apparatus 102 includes user tracking devices (UTD) 104 for tracking users (e.g., firefighters) entering premise 106 under the hazardous environments described above. A premise may be a house, building, barns, apartments, offices, stores, schools, industrial buildings, or any other dwelling or part thereof known to those skilled in the art. In this embodiment, apparatus 102 also includes other functionality such as voice communication and biometric monitoring as part of UTD 104, but in other embodiments these functions may be components or modules that are separate from the UTD 104 or not present at all. In the embodiment described herein, system 100 includes two or more user tracking devices (UTDs) as described in more detail below. However, any number of UTDs may be employed as known to those skilled in the art. Examples of the particular type, construction and mechanisms for mounting apparatus 102 and/or UTD 104 are described in more detail below.

System 100 incorporates mobile device 108 that communicates with a network and central computer system 112 (described below) via the Internet 110. Mobile device 108 is configured to access a portal of data obtained from the biometric sensors as described in more detail below. Mobile device 108 include tablets (e.g., iPad), phones and/or laptops as known to those skilled in the art. The platform, as described in detail below, can be viewed on any type of mobile device 108 such as a phone, laptop, or desktop with proper credentials via a web application. However, any number of mobile devices may be used. Mobile device 108 communicates with cloud 118 to access various data as known to those skilled in the art. Mobile device 108 will function as a command unit as described in more detail below.

System 100 further incorporates central computer system 112 that communicates with a network such as Internet 110 and the central computer system 112 via the Internet 110. Mobile device 108 will access data and the platform for performing the function of the location tracking system described herein (and FIGS. 1-3) on central computer system 112 via Internet 110. In an embodiment, complex computations are processed via the cloud. In another embodiment, these computations are processed locally on the hardware. In a final embodiment, computations are made in both the cloud and on the local hardware. (Alternatively, mobile device 108 may store and process the platform for performing the functions of the location tracking system described herein and may directly communicate with UTD 104.) Central computer system 112 includes one or more servers and other devices that communicate over a local area network (LAN). Servers have conventional components including one or more processors, memory, storage, network interfaces and additional components known to those skilled in the art. Central computer system 112 also communicates with cloud 118 to access various data as known to those skilled in the art.

In one embodiment, system 100 may also incorporate computer system 114 on vehicle 116 (e.g., fire truck) that communicates with mobile device 108 via WIFI, LoRa or Bluetooth Low Energy (BLE) or other communication protocol and communicates with central computer system 112 via Internet 110 as known to those skilled in the art. A vehicle may be a fire truck, fire engine, or any equivalent first responder vehicle or other vehicles known to those skilled in the art for rendering service on premises in hazardous environments.

Mobile device 108 as well as vehicle computer system 114 are configured to receive geolocation data from satellite 118 as known to those skilled in the art.

As described above, apparatus 102 includes UTD(s) 104 for users (e.g., firefighters) entering premise 106 under hazardous environments described above. In one embodiment, two user tracking devices will be mounted on each user, one preferably mounted on a user's head (e.g., on PPE or directly) and the other preferably mounted on an ankle, leg, boot, wrist, or in a pocket of the user. The head-mounted device or module provides orientation while the ankle or leg-mounted device or module provides steps. Additional steps could be obtained from a wrist mounted device. UTDs 104 are also adapted to access geolocation data via satellite 118 via GPS transceiver 120 as known to those skilled in the art. Both UTDs 104 (apparatuses 102) are configured to communicate with mobile device 108 and central computer system 112 via Internet 110 as known to those skilled in the art.

Communication between apparatus 102 and mobile device 108 may be conducted directly between the two components or via central computer system 112 (or vehicle computer system 114) as known to those skilled in the art. This is described in more detail below. In addition, mobile device 108 may alternatively communicate directly with UTD 104 without need for central system 112 and/or vehicle computer system 114.

UTD 104 includes inertial measurement unit (IMU) 122 for measuring and reporting specific force, angular rate, and orientation of the user's body as known to those skilled in the art (i.e., acceleration, velocity and position) using accelerometer 122-1, gyroscope 122-2 and magnetometer 122-3. A pressure sensor 122-4 is also incorporated and used to inform vertical distance (Z axis). In particular, IMU 122 functions to detect user linear acceleration using accelerometer 122-1 and rotational rate using gyroscope 122-2. Magnetometer 122-3 is used as a heading reference. IMU 122 may also be GPS enabled. All three components (accelerometer, gyroscope and magnetometer) are employed per axis for each of the three principal axes: pitch, roll, and yaw. In the present embodiment, IMU 122 mounted on the user's head is used to determine user orientation or direction and the IMU 122 on the user's foot is used to determine the distance in steps along the X, Y and Z axes. In this embodiment, UTD 104 further includes environmental sensors 123 including barometric pressure that helps calculate the relative altitude of the user. In some embodiments, there are additionally toxicity sensors for compounds like carbon monoxide, hydrogen cyanide, nitrogen dioxide, sulfur dioxide, hydrogen chloride, aldehydes, and such organic compounds as benzene. In addition, data collected from various movements and gaits tied to individual operators can train a machine learning (ML) model to better recognize user gait, crawl, level step, and stair transition step movement patterns in a variety of circumstances. In other embodiments, one or more environmental sensors 123 may be separate from UTD 104.

UTD 104 further includes one or more sensors 124 such as ultrasound sensor 124-1 that is used to detect and determine distance between UTD 104 (user) and objects within premises 106 such as walls and doors, which would establish internal configuration. UTD 104 further includes microcontroller 128 and battery 130. This sensor can also be used to verify predicted floor plans in real-time by taking into account user position and distance to boundaries such as walls, doors, windows.

Microphone 132 and headset/earpiece 134 (and radio 133 as described below) are part of apparatus 102. These components are preferably neither part of UTD 104 itself nor its functionality (as shown in FIG. 2). However, these components may be designed to be part of UTD 104 if desired.

Microcontroller or microcontroller unit (MCU) 128 controls the operation of UTD 104 (and apparatus 102) as known to those skilled in the art. MCU 128 receives and processes sensor and other data from sensors IMU 122, sensors 124, biometric sensors 126, environmental sensors 123, ultrasound sensors 124, infrared cameras 129, as well as any other sensors that are part of apparatus 102. MCU 128 integrates communication module 128a to enable data to be sent to mobile device 108. Communication module 128a may transmit data from MCU 128 to mobile device 108 via a LoRa module (board) or any other wireless protocol or techniques such as WIFI, Bluetooth, radio and/or LTE modules (to name a few). In the event communication from any UTD to mobile device 108 or satellite 118 is hindered or blocked due to structural building interference (such as basements, stairwells, or other objects or structural impediments), data transmission may be achieved between multiple users via a LoRa meshing network on the UTDs. In this way, the users may transmit data between and through each other (piggybacking) to maintain communication with mobile device 108 and/or central computer system 112. MCU 128 may communicate with third party systems via Bluetooth or any other protocol as known to those skilled in the art.

Battery 130 provides power to MCU 128 as known to those skilled in the art, MCU 128 and sensors. In one-embodiment, battery 130 also powers the throat microphone 132 and earpiece 134 and other components as needed that are part of apparatus 102. However, in another embodiment, sensors 122 and 124 as well as MCU 116 may be powered independently of microphone 132 and earpiece 134 from other power sources directly integrated into existing batteries on the user's self-contained breathing apparatus (SCBA) as described in more detail below, radio, other PPE, or 3rd party source. Also, apparatus 102 may employ a port for direct charging and/or data transfer or software updates. Alternatively, apparatus 102 may be charged inductively (without port) for weatherproofing and moisture prevention. In another embodiment, charging can be delivered via induction-based coils without the need for a port to further improve ruggedization, weatherproofing, and moisture prevention In this respect, apparatus 102 may be configured to receive software updates over the air. Battery 130 is preferably rechargeable, but it may be the type that can be replaced.

Microphone 132 is configured to receive voice commands and headset/earpiece 134 is configured as an audible device as known to those skilled in the art. In one example, microphone 132 and earpiece 134 are configured to communicate with mobile device 108 via (interface with) directly through MCU 116. Alternatively, microphone 132 and headset/earpiece 134 may communicate with mobile device 108 or through a traditional radio 133 employed by users in hazardous environments such as fires. Additionally, the voice data from the radio 133 or headset/earpiece 134 can be processed as text on the portal on the mobile device 108 and may be done directly through MCU 128.

As described above, apparatus 102 may also include one or more biometric sensors 129 to measure and obtain or collect critical health information of the user. In the example in FIG. 2, biometric sensors 120 are configured as separate component(s) of UTD 104 as these sensors contact the wearer (user) directly such as the wearer's skin. However, sensors 129 may alternatively be part of UTD 104 itself. Biometric sensors are described in more detail below, but example biometric sensors include temperature (body) sensor for measuring body temperature, skin temperature sensor for measuring the temperature under the PPE of the user and a combination pulse sensor and oxygen saturation sensor for measuring heart rate and oxygen saturation of the user. In some embodiments, galvanic skin response, blood pressure, EKG sensors may also be placed. A heart rate sensor may also be employed. Any type and number of sensors may be employed to achieve desired results for various environments. Data from the biometric sensors are transmitted via JSON architecture to a portal on mobile device 108, but any other architecture may be used as known to those skilled in the art.

In this embodiment, apparatus 102 further includes one or more infrared (IR) cameras 126 that are connected to the MCU 128. IR cameras 126 are used to create images and capture other data and transmit to mobile device 108 or computer via MCU 128 as described in more detail below. IR cameras 126 (and any other cameras) are configured as a part of UTD 104 in this embodiment, but alternatively, it may be a separate component from UTD 104. Apparatus 102 may include other cameras as known to those skilled in the art.

In one embodiment, biometric sensors 129 and/or microphone 132 are mounted on a user's neck as it is a point for biometric data (carotid arteries) collection and sound detection. In one example, UTD 104, biometric sensors and/or microphone 132 may be integrated as part of apparatus 102, in one piece or component. Alternatively, sensors may be mounted separately (from themselves and/or microphone). Both the biometric sensors may be mounted on other user body parts provided they offer desired data measurement/collection. Microphone 134 must be in proximity to a user's head to provide adequate sound detection such as on the SCBA or fire hood, e.g., to detect voice commands for clearing rooms, mayday or other commands, etc. (Voice commands may be issued directly on the portal.)

Headset/earpiece 134 is preferably mounted on or in a user's ear, but headset/earpiece 134 may be mounted on the user at other locations in proximity to the user's ear (for hearing detection). An example earpiece is bone conductive or otherwise but this earpiece requires contact with or slightly forward of the user's ear.

The headset/earpiece may be a low power draw earpiece and duplex throat microphone with the ability to press a button associated with the microphone to initiate talking. This button to activate the microphone can be located on the neck piece or on the earpiece for ease of use. In addition, in some example embodiments, push to talk or pinch to talk buttons may be utilized. For example, such a button may be located proximate to the neck to allow the user to easily enable communication. In some embodiments, a pinch-to-talk button utilizes one or more mechanical switches. In other embodiments, one or more RFIDs and sensors are embedded in the fingertips and neck. In some example embodiments, integrated adaptive noise cancellation is included in the system 100. This communication system is preferably hands-free, noise-canceling, and allows for seamless communications between the operator and additional team members via radio transmission.

In another embodiment, the biometric sensors 129 are mounted on a user's wrist for ease of use and to avoid discomfort and potential strangulation. In addition, other third-party biometric devices may be used with system 100 such as those mounted on arms, wrist and core (i.e., wrapped around chest or stomach).

Notifications of abnormal thresholds may be triggered and shown. LED alerts may be employed for hardware issues or biometric data and/or threshold analyses abnormalities (e.g., temporary spikes or prolonged time spent above thresholds). Voice analysis and commands may trigger alerts. Vibration, audio alerts or other notifications may be employed. Thresholds and states may be set by an individual user/operator. Voice to text functionality and command to voice (via portal) may be employed.

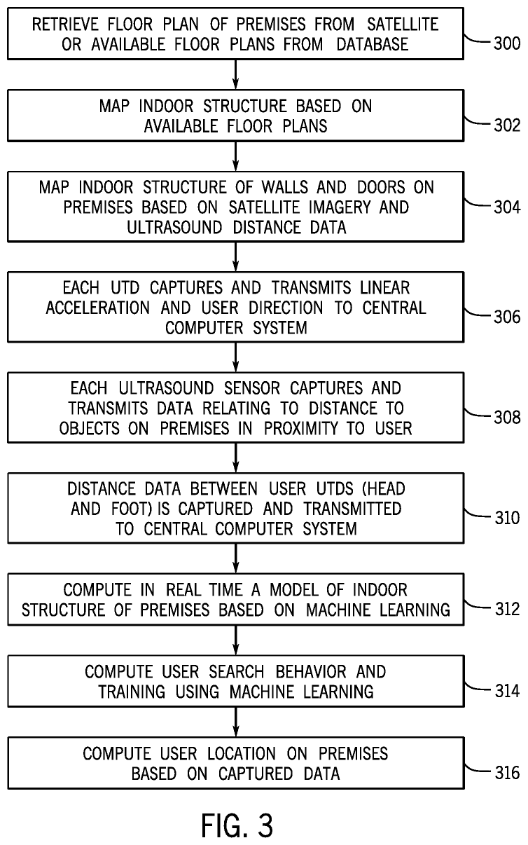

FIG. 3 depicts a flow diagram of the platform steps for performing the function of the user location tracking system of FIG. 2. In brief, the platform generates floor plans, tracks users (e.g., first responders—firefighters) in 3D on premises (e.g., multi-story building along X, Y, Z axes), and identifies users as they enter and exit the premises or incident area. In addition, the platform notifies the incidence commander of detected maydays from falls or abnormalities via health and environmental alerts. The platform is compatible with all existing connected technologies on the fireground and serves as the primary tool for pre-planning as well as consolidating all the information needed for report-outs. The platform steps below represent a high level process of data collection, analysis and functionality during pre-planning, incidence and post-incidence phases of platform deployment. Note that in this embodiment described below, the platform is stored and operated on a central computer system as needed by and in connection with a mobile device. In another embodiment, the platform and data may be stored and operated on the mobile device and/or cloud itself without a central computer system.

Execution begins at step 300 wherein the floor plan of the premises is retrieved from satellite imagery and/or available floor plans from a database. Specifically, satellite images and floor plans are obtained from sources such as Zillow, Redfin (for example) which will be processed by the machine learning pipeline to ultimately create likely structure floor layout as described below. Composite premises floor plan images from all sources are stored in a database within the central computer system or in the cloud. Alternatively, data may be stored on the mobile device and cloud without any central computer system.

Execution proceeds to step 302 wherein the existing internal configuration layout is displayed. In some embodiments, the internal configuration may be altered to enhance readability. These floor plans can be pre-planned provided by the Fire Department, Municipality, or other publicly available sources such as Zillow or Redfin.

Execution proceeds to step 304 wherein, in the event available floor plans are not available from third-party sources, the indoor configuration of walls and doors on-premises are generated using a machine learning model based on satellite imagery from sources such as GIS satellite data or from apps such as Google or Microsoft Maps. When no floor plan is publicly available from sources such as Zillow or Redfin for example, the platform utilizes a machine learning (ML) model to predict the layout of the floor plans. This is accomplished through identifying outside constraints (e.g., walls, windows, doors, roof shape, number of stories) collected from satellite imagery (e.g., Google maps, street view or GIS imagery). These constraints are then loaded into a model that is then pulled from the database of the other floor plans to make a prediction of the internal layout.

Execution then proceeds to step 306 wherein linear acceleration, velocity, position and directional data are captured by user mounted UTDs and transmitted to the central computer system to help determine localization (of user). Once a user enters a premises, GPS accuracy and availability may be hindered or blocked so GPS access is terminated in this embodiment. In some detail, the satellite is used for GPS outside the premises and switches to local hardware when the user enters the premise structure. Specifically, the platform (location tracking) switches from GPS to UTDs 104 and mobile device 108 (local hardware) or vehicle computer system 114 once the user enters the premises. GPS is no longer relied upon when inside premises. The platform, described below, thus detects user entry and switches as described in one of two ways. In the first instance, detection occurs when a boundary of the premise structure is actually passed (GPS) and the user enters the premises. In the second instance, detection occurs when the GPS signal “jumps” around indoors, as time to return (signal) is getting significantly elongated as known to those skilled in the art. UTDs and other available data are used to user location tracking as described herein. In the current embodiment, the UTD mounted on a user's helmet, mask, or other embodiment located near the head generates acceleration, velocity and position data (including orientation or direction data) and the pressure sensor will generate Z-axis data. The UTD mounted on the user's foot (e.g., boot), ankle, pocket, or wrist generates step length (X, Y, Z axes) as well as steps up or down between floors (distance) and Z-axis coordinates.

Execution proceeds to step 308 wherein ultrasound sensor data is captured and transmitted to the central computer system. The sensor data relates to the distance from objects in proximity to the user (e.g., firefighter) on premises.

Execution proceeds to step 310 wherein the distance between UTDs (head and foot) is captured and transmitted to the central computer system. The distance data helps to determine the physical status and/or position of the user such as a fallen or collapsed user. The distance data may be captured by direct communication between UTDs or over a network (Internet 128).

Execution then proceeds to step 312 wherein a model of the indoor structure is computed based on a machine learning model. Specifically, floor plan images will be used to create a machine learning tool(s). Training data will be increased with floor plans from satellite images and images through sourcing of publicly available floor plans such as via Zillow, Redfin (for example) which will be processed by the machine learning pipeline to ultimately create a likely structure floor layout. In addition, neural networks may be used for image segmentation and for distinguishing between buildings, road and other features on satellite imagery. In person (user) data will also be inputted and merged to improve incident command capability to gain insight into the internal building structure to guide user (firefighter) deployment and navigation as described herein and below.

Execution then proceeds to step 314 wherein user search behavior and training are used in the machine learning model to predict user location and direction.

Execution then proceeds to step 316 where user location on premises is determined along with predicted direction based on captured data such as building structures, mapping data, ultrasound data and user behavior. For example, if the sensors indicate the user is moving to the right and then left but based on user behavior and training, the system platform determines that the user may be moving to the right only based on user behavior and training (e.g., firefighters may be trained to move right along a wall during a search). That is, if a majority of sensors data indicates movement to the right, and according to the floor plan, a right-hand search is the preferred method of a user search method, then movement to the right is confidently indicated.

The process steps above may be performed in a different order or with additional steps as known to those skilled in the art.

While not specifically called out by the steps above, the platform for performing the functions of the tracking system described herein enable communication between user UTDs (on multiple users) in order to piggyback onto a network in the event communication between a UTD and mobile device is hindered or blocked. LoRa module meshing is an example protocol employed to enable such communication. The platform also enables access to data from other sources via one or more APIs (for example) such as fireground or fire station computer systems for full accountability of the users (e.g., firefighters) on and off premises and other vehicles rendering service. The platform also enables access data from third party devices such as Apple watch and Fitbit (as examples) via Bluetooth meshing or other protocols of communication.

### UTD Type, Construction and Mounting

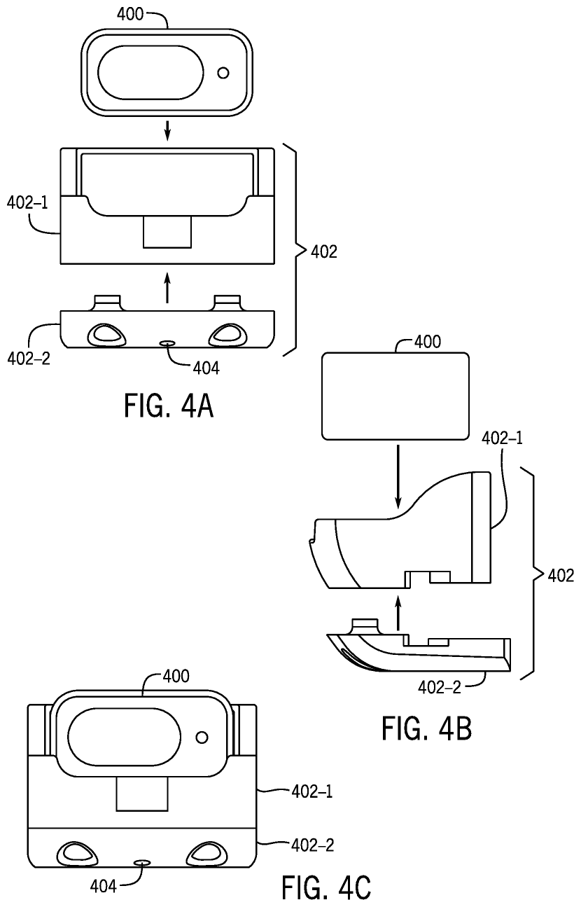

As indicated above and in summary, as shown in various embodiments in FIGS. 4A-4C through FIGS. 18A-18D, UTD 104 and/or other components in apparatus 102, is mountable to existing (conventional) PPE typically using a mounting mechanism referred to as a mounting accessory as described hereinbelow. The UTD housing or enclosure allows for an open interior that can be filled with the various sensors, tracking components and other electronics described hereinabove as well as potting material in certain embodiments as potting is beneficial for ruggedization, weather and waterproofing. The UTD 104 may be a two-piece module or component in which one piece is an enclosure used to hold the sensors, tracking components and/or other electronics and the other piece is used as the mounting accessory. These two pieces are clamped shut. In other embodiments, the mounting accessory and enclosure are configured as one integral component.

For each component of PPE, the mounting accessory as described above clamps or attaches onto the outer edge of the equipment. For some PPE, protective equipment's existing mounting accessory points are utilized. In other embodiments, the mounting accessory is configured to clamp around a bezel of an outer enclosure or the edge of a surface of a helmet or other head PPE or to clamp onto the edge (lip) of a helmet. Alternatively, the mounting accessory is configured to be inter-woven into existing webbing systems like Pouch Attachment Ladder System (PALS), slotted into existing rail systems like dovetails or reverse dovetails like those found on the Future Assault Shell Technology (FAST) helmet, attached to existing attachment points like the M-LOK system developed by Magpul Industries, or other locations or edges on the PPE. In other embodiments, the mounting accessory is configured to clamp around the rail of the helmet configuration currently used by European firefighters as well as ballistic helmets used by the military and law enforcement.

These designs are generated from a 3D scan of the PPE or existing CAD files, or via an iterative process of measuring and 3D printing to test fit. As a fitted contour of the protective equipment mounting points is required, each outer enclosure is unique to each model of PPE. In some embodiments, the inside of the accessory mounting point(s) contains wiring and connectors to allow for communication and power to transfer between modules, which can provide feedback indicating that modules are correctly attached into the mounting accessory. This can provide haptic feedback on a reliable connection as well as begin a stream of data via wireless connectivity, which is detailed below. Power may be drawn from existing batteries already on the SCBA mask, helmet or other sources.

### Module Design Material.

To meet user demands, the module housing or enclosure is constructed of durable, rugged, and environmentally resistant materials. These materials create a hard outer shell to protect the user and help ensure that sensors, tracking components and/or other electronics are safely housed and have a reliable connection. In some embodiments, a metal, such as hardened aluminum for example, can also be embedded into the polymer or placed on the inside edge to further improve the structural integrity of the enclosure. In some embodiments, thermoplastics like Acrylonitrile Butadiene Styrene (ABS), Polyethylene Terephthalate Glycol (PETG), or Polylactic Acid (PLA) are used to build the enclosures. Additional layers of reinforcement weigh more, but also increase durability and resilience for extreme environments. Use of strong, but lightweight materials helps to ensure that the module(s) remain light enough to reduce strain on the helmet and mask or wearer's neck and upper body.

### Module Potting Material

In one embodiment, a potting material is used in conjunction with the module(s), tracking electronics and mounting accessories to help ensure that the electronics inside are waterproof, temperature resistant, impact-resistant, and intrinsically safe. The potting material is poured into the enclosure post assembly or brushed on or poured over connection points and some or all electronics are encased by the potting material as its sets. In addition to protecting the electronics, the potting material improves the overall structural strength of the assembly by providing a normal force against strain, stress, torsion, and/or impact. The potting material may also act as an adhesive, keeping both the top and bottom portions of the enclosure together. In some embodiments, the potting compound is polyurethane or silicone, to avoid solder fatigue through a lower glass transition temperature on surface mount circuit boards. In other embodiments, multiple formulations of potting compounds are delivered in different layers to allow for mechanical characteristics where needed. The module preferably maintains wiring and connectors inside that is connected to external power sources (existing batteries) that may already be present on SCBA or helmets.

The material throughout the module is preferably hypoallergenic and can be sanitized between uses, including via a soak detergent, such as one frequently used by the United States Department of Defense. A potting material allows the modular electronics within the enclosure to be impact, water, and fire-resistant. The module should be rated to survive washing and cleaning materials.

### Module Batteries and Charging

Battery or batteries as described herein may be charged via a charging port located on the edge of the module or other locations. The battery on a removable camera (or other modular sensors) can also be charged via a similar interface to that of the mounting system. This charging system may, for example, be compatible with standard commercial power tool charging apparatus. In one embodiment, a magnetic connection system can be utilized to provide a wired connection for charging without exposing open charging ports to the outside environment.

In another embodiment of the module(s), induction-based charging can be utilized to avoid the need for an exposed charging port. The induction-based charge includes a coil system integrated on both the module and a removable camera module.

Additional power sources can be added and removed as needed. Current industrial respirators incorporate an elastic or cloth strap to securely fit around the head or ease the use of carrying or slinging. To this system, auxiliary batteries and an interchangeable and modular battery system can be integrated, to provide for various power draws. The modular battery may optionally incorporate the ability to self-charge through the integration of solar panels, heat inducting coils, or mechanical energy motion capture. These modular components are also removable for ease of replacing damaged parts or cleaning.

### Module Wearer/User Notification System

In one embodiment, multicolor LEDs placed in the peripheral of a user's (mask) visor field of view allow for the delivery of actionable insights to the user.

These LEDs can be controlled either from a user interface from outside the premises by others detailed below or in conjunction with threshold alerts built into the coding of the visor itself. These thresholds can be altered from (and additional thresholds can be added) the user interface. In one embodiment, stencil-based icons can be backlit by these colored LEDs.

The displays present sensor readings in the form of icons and alerts that may include, without limitation, information such as blood pressure, heart rate, pressure leak alert, CO2 build-up alerts, team biometrics, and a shared compass. These icons are preferably color coordinated and/or with distinguishable shapes to account for the inability of the eye to focus on objects close to the face. An electric circuit can be used to indicate when connectivity is lost and for what period of time.

Additionally, the alerts system can be used to identify hazards, such as the detection of hazardous gases. An environmental sample can be collected via sensors in the module and the information can be used to estimate the amount of containment that has entered the environment. Additionally, face seal pressure can be monitored via an air pressure sensor or a carbon dioxide sensor, discussed below in the module section. Users can be notified of small sustained leaks. For example, the user may be alerted via a color icon or LED on the heads-up display when this pressure seal is lost, according to one embodiment.

In another embodiment, the visor is embedded with thin-film electronics that are opaque and used for electrical display. In one embodiment, this display is a thin film OLED display mounted on the inside edge of the visor near one of the eyes. In another embodiment, this system projects light into an etched portion of the visor(s) acting like a screen displaying an image cast by a projector. The features of the Heads-up Display (HuD) include the display being built into the visor. In either case, a mechanical attachment may be added to the module to allow for the visor to be placed near the wearer's eye. This attachment is preferably low profile and conforms to the contours of an inner visor portion, thus allowing the user to wear prescription eye lenses while still maintaining a pressure seal against an outer visor portion.

### Module Optical Sensors

One or more optical sensors, cameras, night vision, and/or infrared lenses may be mounted on the edges of the accessory mounting point to record and enhance the wearer's perspective. The wearer may, for example, view any images or videos produced by these optics in real-time or as past recorded events. This information can be displayed on the integrated heads-up display or be utilized in the real time mapping and localization task. Open sensor pins may be provided for the integration of various sensors or modules to aid in the adaptation of various optical modules, such as a flashlight. A custom suite of compatible sensors can be integrated into the system that changes the orientation of the heads-up display. There are multiple streaming options via Bluetooth, WIFI, or LTE, for example. An IR array can provide thermal imagery to augment repair and maintenance.

In an embodiment, the system camera(s), (e.g., IR camera) may be removable and may include its own battery module and microcontroller unit with wireless connectivity. This allows the user to use the camera as a system independent from the rest of the module in order to record footage of areas outside of the direct line of sight, e.g., during avionic maintenance and repair.

The removable camera module also features a toggleable flashlight with adjustable brightness. The rest of the module contains, for example, an auxiliary microcontroller and battery that allows for the additional sensors to continue to function when the camera system is removed. These two systems are preferably powered independently and both systems' microcontrollers contain protocols to communicate with each other or with a separate controller. Alternatively, the camera module can be removable but with a retractable cable.

### Module Location and Environmental Sensors

An integrated GPS sensor, receiver or transceiver as described herein preferably includes a sensor with a low warm-start time and may utilize GPS, Global Navigation Satellite System (GNSS), Quasi Zenith Satellite System (QZSS) and/or Satellite Based Augmentation System (SBAS), for example. By utilizing the accelerometer of the IMU 122 and GPS-loaded data onboard, a compass indicator can be populated. Additionally, the module may include electronics to allow the user to ping or mark shared objectives or locations via the use of a guided laser.

Temperature sensors as part of environmental sensor 123 are included to read a wide range of temperatures. Additionally, a temperature reader can be integrated using a laser-guided infrared reader to allow point readings at a distance. An infrared array system can take temperature readings without the need for contact-based readings.

Pressure sensors are preferably included, on an exhalation vent of the gas mask visor on an inside surface of the enclosure or on the pressure regulator of an air tank itself to notify the user of how much air is remaining and, in some embodiments, to assist in extrapolating time remaining from past usage. Additional environmental sensors, such as Geiger counters and/or air quality sensors, can measure ionizing radiation as well as volatile organic compounds in the air. The pressor sensors may be separate or part of the barometric sensors 129 or environmental sensors 123 for example.

Additionally, one embodiment includes sensors for measuring CO2 buildup in the system, to detect and warn operators of a kink in the air supply hose or any other mechanical issues before such issues affect breathing. These notifications can be triggered for a specific readout combination between various environmental sensors such as the pressure sensor or carbon dioxide detectors, or from the electronic-mechanical or magnetic seal between the module and fabric hood. The CO2 sensors may be separate or part of the barometric sensors 129 or environmental sensors 123 for example.

### Module Wiring, Connectors, and Charging

In one embodiment, mechanical, electrical, or electro-mechanical connectors are utilized between microcontroller (MCU), breakout, and battery. All wires and connectors are environmentally ruggedized to account for water, heat, and impact. Alternatively, transductive mounts (e.g., with magnets), or direct soldering, can be used. For some breakout boards, custom adapter boards may interface between I2C, serial communication and PWR protocols.

The system 100 is preferably built in a modular and extensible framework—i.e., sensor packages are modular and variously sized with consistent connector points. The described and illustrated connector provides both power and data connectivity as described herein. In one example embodiment, the connector includes 3.3v, ground, SDL, and SCA connections, but other embodiments could include other data protocols in addition to 12C. Further, the connector is preferably consistent/compatible across the modular sensor ecosystem, allowing the array of sensors to click in with a mechanical design as described herein for haptic feedback which also locks the sensors firmly. In some embodiments, mechanical swivels are built-in on the top of the connector, while in other embodiments, bolts or rigid connections (e.g., welds) hold the modules firmly in place.

Some connectors could utilize magnetic connection points as described hereinbelow, such as between the optical sensor suite and the rest of the outer enclosure. A combination of mechanical and/or electrical feedback is given to the user via audio or visual, such as via a heads-up display, to provide confirmation of correct interfacing. This haptic feedback can also alert the user when connections have not been made.

### Biometric Sensors

Biometric sensors 129 interact directly with the wearer as described above. A housing or enclosure houses wires and/or electrical sensors that are accommodated to be easily removed for modular replacement and maintenance on the electronics. The modules are preferably waterproof/water-resistant and/or impact-resistant. The housing houses embedded health sensors to measure and track biometric data such as heart rate and blood pressure. These biometric sensors preferably make physical contact with the skin and are worn around the neck of the wearer or other areas. In one embodiment, a housing for the biometric sensor package is mounted on a throat microphone, which helps to apply pressure to the microphone to maintain contact with the neck for better voice pickup. In some embodiments, this housing or enclosure is made of Kevlar printed material, while in other embodiments, it may be Acrylonitrile Butadiene Styrene (ABS) or Polyethylene Terephthalate Glycol (PETG) for flexibility, or a combination of materials (including others not listed here).

In some embodiments, the biometric sensor components or modules are mounted (and removable/detachable) via hook-and-loop fasteners (e.g., VELCRO™), buttons, loops, magnets, onto a fabric median to allow for a more reliable fit or which moisture. The fabric material can contain moisture-wicking and antimicrobial properties, such as a polypropylene fabric with silver fibers to provide antimicrobial properties and conceal the wearer from infrared cameras. The stitching patterns in the fabric hood are preferably optimized for strength, with Kevlar thread being selected for heat resistance in some embodiments. In one embodiment, a plastic loop retains a throat microphone module. In other embodiments, a Kevlar loop serves the same purpose.

The electrical system of the biometric sensors and communication equipment (e.g., headset earpiece) may utilize flat wiring and nonpolarized connectors to allow for ease of assembly as well as maintenance and increased comfort and reliability. Connectors and pins can also be flexible in nature to ensure connectivity. The electrics may be embedded into the biometric sensor and/or communication equipment housing to better manage wire housings and reduce snag risk.

In one embodiment, system 100 can be powered via an internal or external battery that can be swappable or removable as described above. Connector points in the electric system may also be coated in hydrophobic and flexible polymers to ensure resilience against sweat, oil, stretching, and pinching.

Biometric sensors may incorporate pulse oximetry sensor circuitry that can measure heart rate and provide feedback to both the operator and to other parties. This data can be used to identify potential health risks and take precautionary measures. Sensed pulse oximetry data can stream over multiple connectivity stacks such as Bluetooth, LTE, WIFI, and/or directly over radio via narrowband. Pulse oximetry and heart rate data may be based on an easily additive or subdivisible JSON architecture, utilizing string-based data packets with not more than 10 bytes per packet, for example (compared to the rest of the system, which preferably is not more than 200 bytes per packet). Various thresholds and transmission rates can be determined to help reduce the flow of data streaming as necessary. In one embodiment, there is a discrete battery built into each module, which allows for independent data collection outside of the system.

Alerts based on biometric sensor data can be displayed via an integrated head-up display, such as via colored icons that can alert the wearer of potential health risk for their biometric data as well as the biometric data of other wearers.

### Mounting Accessory-Mounting to Existing Respirator

There are generally two main types of respirators—(1) air-purifying respirators that remove contaminants from the air via filtration system and (2) air-supplying respirators, which provide a clean source of external air (also referred to as Self-Contained Breathing Apparatus (SCBA) as described above).

The facepiece for typical respirators will cover either (1) just the mouth and nose in order to ensure a respiratory seal or (2) the entire face with a transparent visor. While the source of air is different for each of these types, i.e., self-contained or continuously filtered, both respirator types must provide an adequate seal on the user's face to ensure adequate ventilation through the respirator so that the user can breathe safely. For respirator types having a full or half face visor or facemasks (referred to herein as “visor”), the visor can impair vision and limit the productivity of the user.

Mounting points may be built into the structure of the respirator visor itself for certain commercially available respirators, either through side mounts or through direct screw holes for accessories. The location of these mounts tends to be on the outer ridge of the visor as described below. In one embodiment, the add-on disclosed herein is attached directly to those locations. In other embodiments, custom-designed adapters are utilized to clamp to the existing respirator structure. UTD of apparatus 102 is designed to clamp onto the outer edge as mounting points. Thus, the respirator's seal or gasket, which provides the fundamental function of the respirator, is unaltered and no bolts or screws are utilized, except perhaps with respect to existing mounting points. In one embodiment, the mounting accessory includes two separate pieces that connect at the chin. In another embodiment, the mounting accessory consists of a single piece. In one embodiment, the data and power connection is at least partially inside of the outer housing or enclosure and is sealed in by a potting compound. In another embodiment, adhesive could be used to secure the outer enclosure mount to the visor.

### Mounting Accessory Mounting to Existing Tactical Helmets

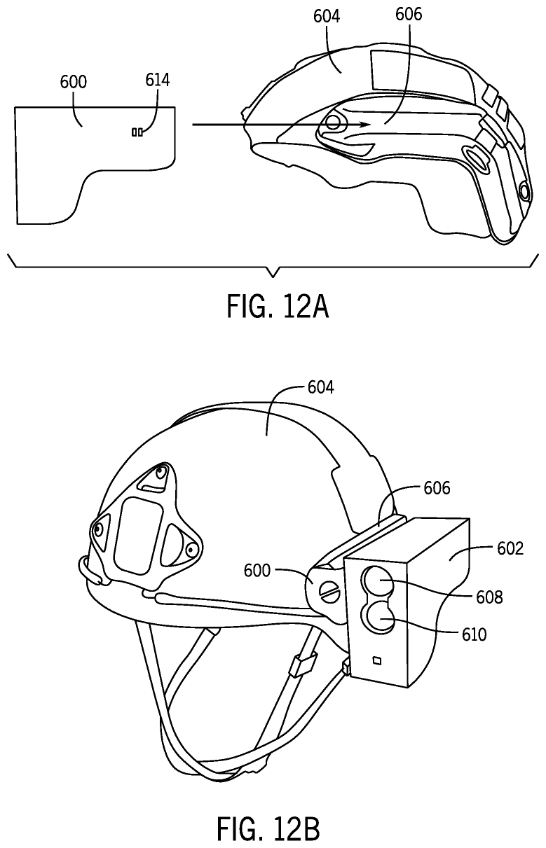

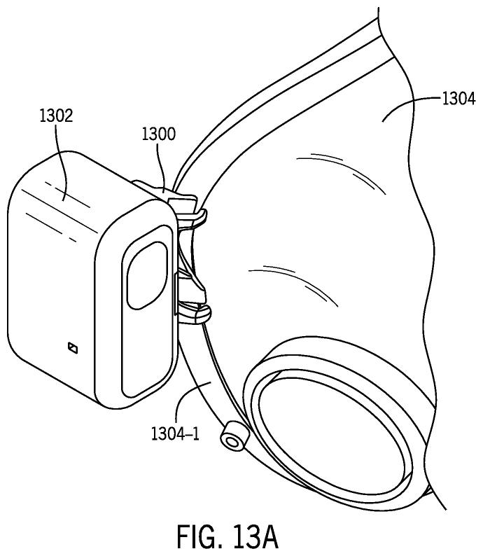

Some commercially available protective helmets (such as ballistic helmets) feature rail mounts for the addition of mountable accessories. This rail mount system features a reverse dovetail infrastructure that allows t-rail connections to be securely made. In some embodiments, the module features a t-rail extruding from the edge of the enclosure to slide in place to mate with the reverse dovetail system. FIGS. 12A-12B depict views of an example of a user helmet with this t-rail and dovetail mating structure. In another embodiment, a magnetic mounting infrastructure that also features an extruded t-rail first slides into the reverse dovetail infrastructure. The module then mates with magnetic mounting points found on the edge of the accessory mounting point. This embodiment allows for sensors to be easily removed while still utilizing existing reverse dovetail infrastructure.

Some commercially available ballistic helmets do incorporate existing rail mounting points. In another embodiment, a fabric strap can be wrapped around the outer edge of the helmet and serve as a rigid mounting infrastructure for an mounting the mounting accessory. This mooting accessory can be woven into the fabric, or on a mechanical mounting infrastructure to allow for the strap to securely hold the mounting accessory into place. The module can then be mounted either through the reverse dovetail and t-rail mating or magnetic connection discussed above. Additionally, placement of an LED indicator system may be located on the edge of the user's peripheral vision to avoid adding to the cognitive load of the wearer/user. Weight of the added module can be mitigated with additional module attachments to the opposite side of the helmet or balanced with additional accessories such as a flashlight.

Example modules, mounting accessories and mounting to various PPE appear in FIGS. 4A-4C though 18A-18D.

FIGS. 4A-4C depict various views of a module 400 that is configured to be mounted magnetically to an example mounting accessory 402. In particular, FIGS. 4A and 4B depicts front and side views of module 400 and mounting accessory 402 in an exploded configuration. Specifically, module 400 is magnetically attached and fitted within mounting accessory 402. Both module 400 and top bracket 402-1 incorporate positioned magnets to facilitate attachment and securement. Mounting accessory 402 comprises top mounting bracket 402-1 that receives module 400 and bottom mounting bracket 402-2 that is screwed to top mounting bracket 402-1. Bottom mounting bracket is clamped tightly to a brim on a helmet or other structure of PPE via a clamping port 404 on bottom bracket 402-2. FIG. 4C depicts an assembled configuration of module 400 and mounting assembly 402.

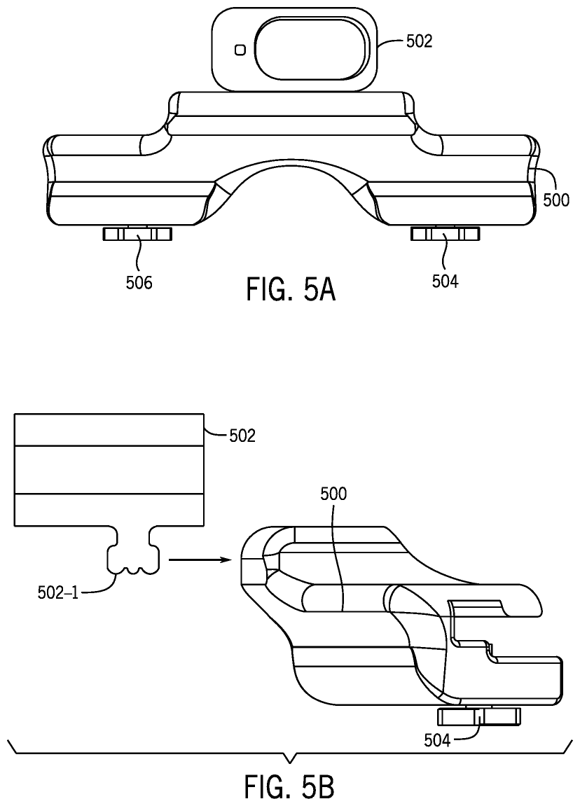

FIGS. 5A-5B depicts various views of another example mounting accessory 500 for a module 502. In particular, FIG. 5A depicts a front view of mounting accessory 500 and module 502 installed within mounting accessory 500. FIG. 5B depicts a side exploded view of mounting accessory 500 and module 502. In this example, mounting accessory 500 employs two screws 504 and 506 to clamp onto a brim of a helmet (PPE). Module 502 is attached to mounting accessory 550 by sliding a projection 502-1 into a track 500-1 or opening on mounting accessory 500.

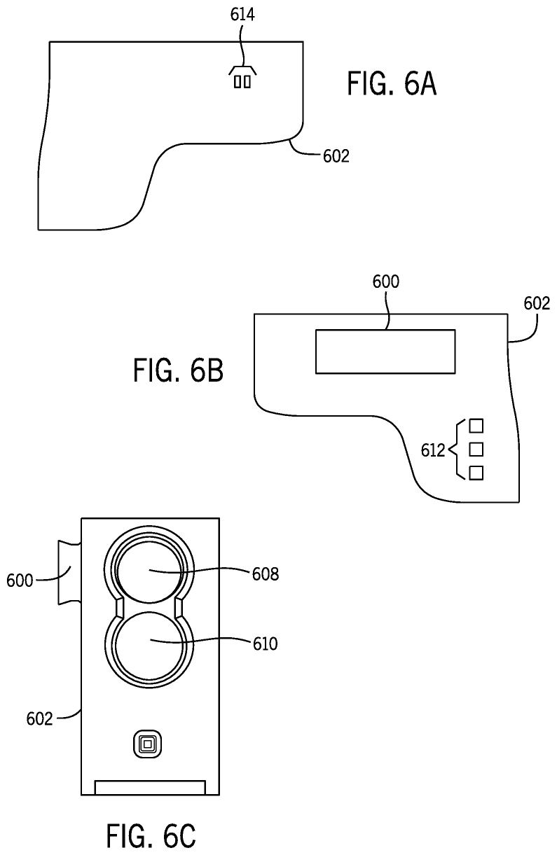

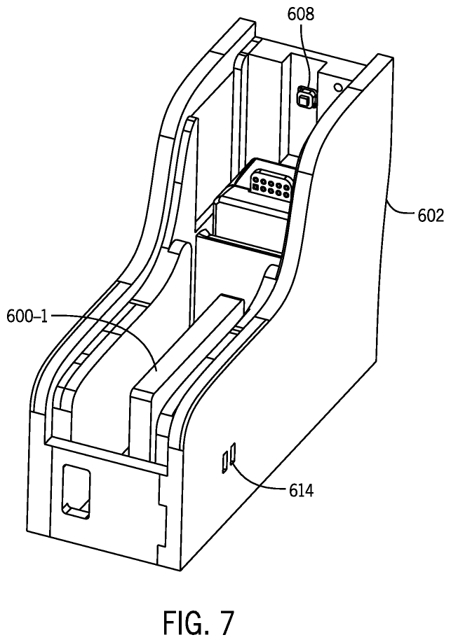

FIGS. 6A-6C depicts various views of another example mounting accessory 600 that is configured to mount module 602 to a FAST helmet 604 (FIGS. 12A-12B). FIGS. 6A and 6B depict side views of mounting accessory on module 602. Mounting accessory 600 is screwed into module 602. FIG. 6C is a front view of both mounting accessory and module 602. Mounting accessory 600 is slotted into existing rail systems reverse dovetails 606 described above and shown in FIGS. 12-12B. A user/wearer slides mounting accessory 600 within rail 606 of FAST helmet 604. Module 602 is thus angled outwardly. Module 600 includes IR camera 608 and light detection and ranging sensor (LIDAR) 610. LEDs 612 are also shown for LED alerts. Inlet ports 614 for environmental sensors are also shown. FIG. 7 depicts an inside view of module 602 wherein several components are shown including IR camera 608 and microcontroller 600-1 as described herein.

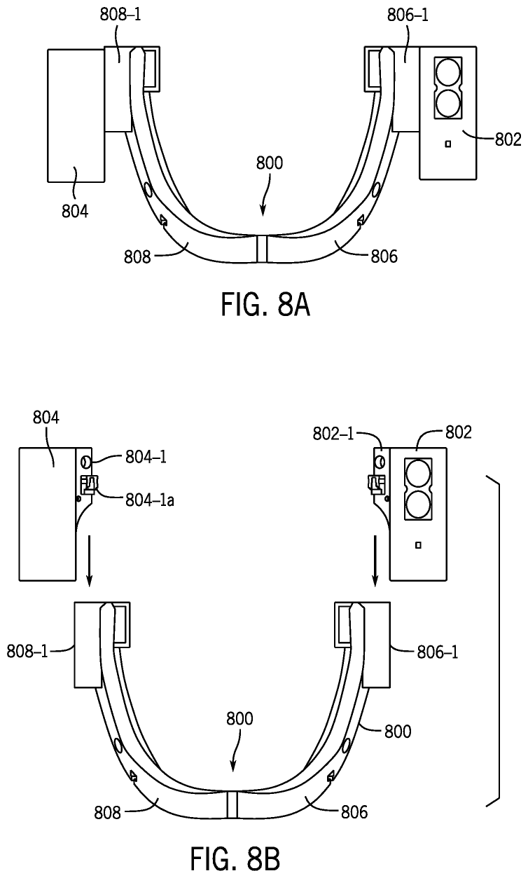

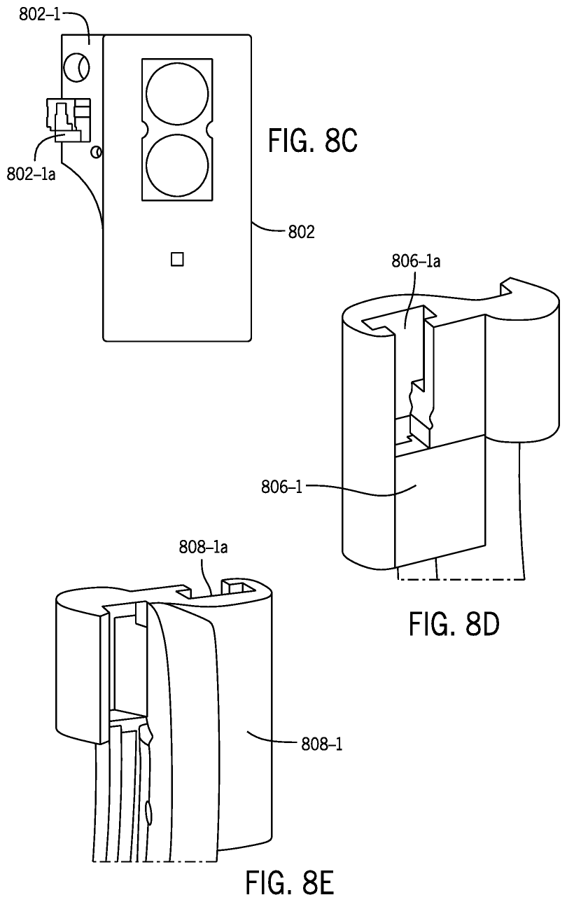

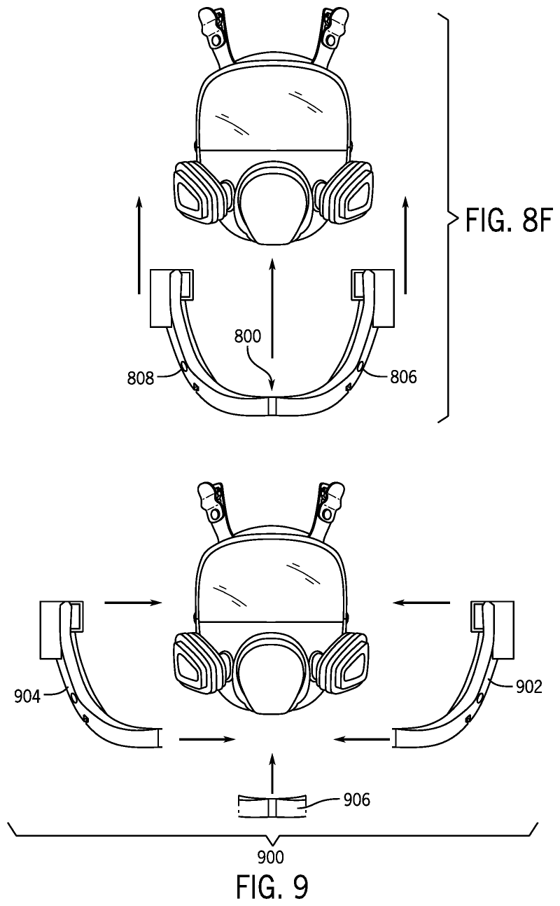

FIGS. 8A-8F depict various views of another example mounting accessory 800 and/or modules 802, 804. Specifically, FIGS. 8A and 8B depict from views of mounting device 800 and modules 802, 804 in post and pre installment configurations. FIG. 8C depicts a side view of a module. FIG. 8F depicts the mounting accessory 800 before mounting to a user's mask.

Mounting accessory 800 is configured as a single piece with two mounting arms 806, 808. These arms are designed to correspond in shape to a user's mask 810. Mounting arms 806, 808 are configured to slide or snap onto user's mask 810 as shown in FIG. 9A. In this embodiment or example, module 802 is configured as a module with IR, LIDAR and IMU and module 804 is configured as a power module that incorporates a battery as well as MCU. However, these modules and their contents may change as desired. In these examples, modules 802,804 are swappable.

Mounting accessory 800 is configured with mechanical ridges as a rail system to ensure proper alignment and reliable mounting modules 802, 804 as described in more detail below. Specifically, modules 802, 804 include protruding sections 802-1, 804-1 that extend from the end top end thereto and latching mechanisms 802-1a, 804-1a.

FIGS. 8D and 8E depict an enlarged view of the clamp or clamping mechanism 806-1, 808-1 that is preferably molded onto arms 806,808 for receiving modules 802, 804. A part of the rail system, clamps 806-1, 808-1 incorporate rectangular slots or cavities 806-1a, 808-1a, respectively that correspond in size and shape to module protruding sections 802-1, 804-1 that extend from the end top end thereof. Cavities 806-1a, 808-1a include corresponding ridges to enable a latching mechanism 802-1a, 804-1a to securely slide and fit with clamps 806-1,808-1. In other embodiments, cavities 806-1a, 808-1a may have desired shape so long as the corresponding in shape to receive and lock protruding sections 802-1, 804-1 in place.

FIG. 9 depicts a view of another example mounting accessory 900. Mounting accessory 900 is the same functionally as the embodiment in FIGS. 8A-8F but mounting accessory 900 is configured as a two-piece structure as shown with arms 902, 904. This embodiment may be used to attach to existing (masks) visors. Clamping mechanism is used to clamp the two-piece arm structure as known to those skilled in the art.

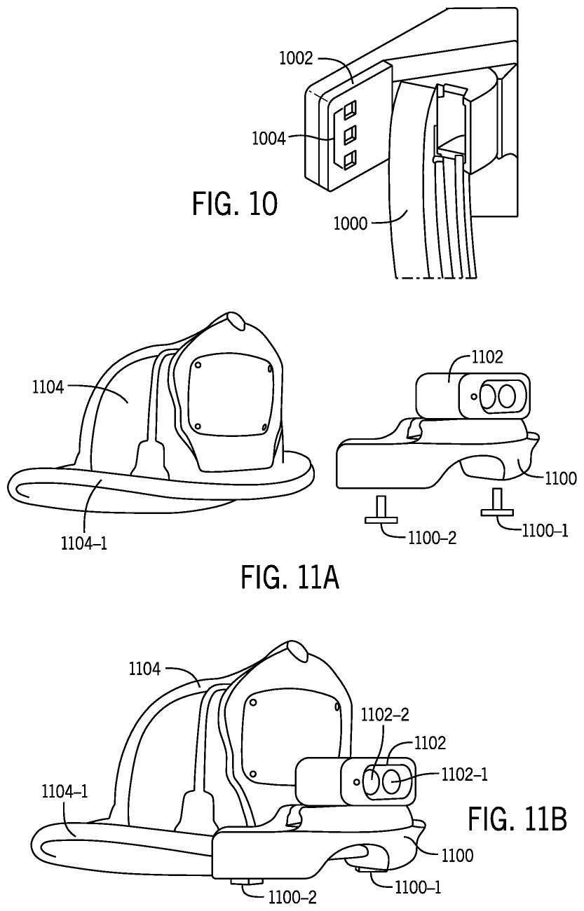

FIG. 10 depicts a front view of another example mounting accessory 1000 for module 1002. Module 1002 is friction fit on mounting accessory 1000. Module 1002 includes three LEDs 1004 as shown and are mounted in a manner that is viewable in the operator's (wearer/user) view.

FIGS. 11A-11B depict front views of another example mounting accessory 1100 and module 1102 in pre and post installed configurations. Mounting accessory 1100 is configured to mount module 1102 to a firefighter helmet 1104 along brim 1104-1 thereof. Mounting accessory 1100 is clamped via screws 1100-1, 1100-2. Module 1102 includes LIDAR 1102-1 and IR 1102-2 as well as environmental sensors inside.

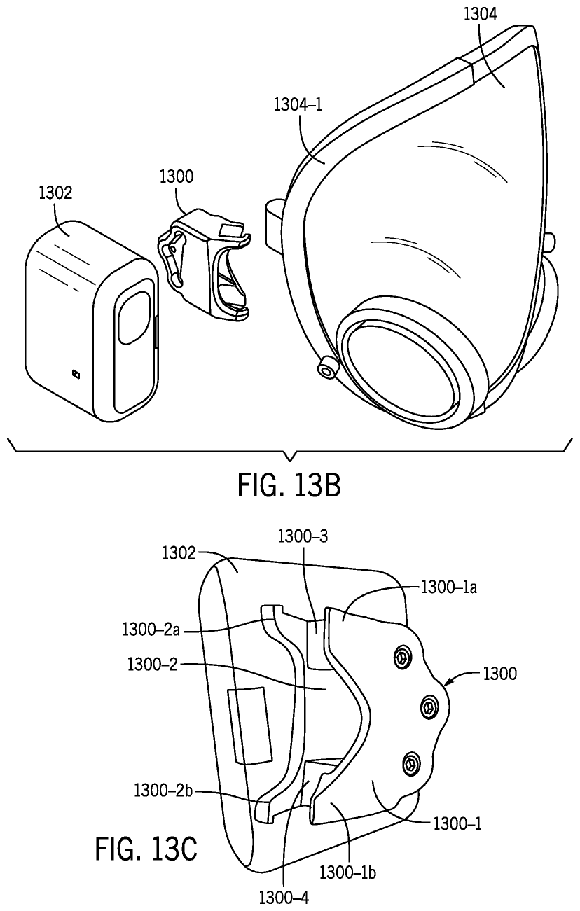

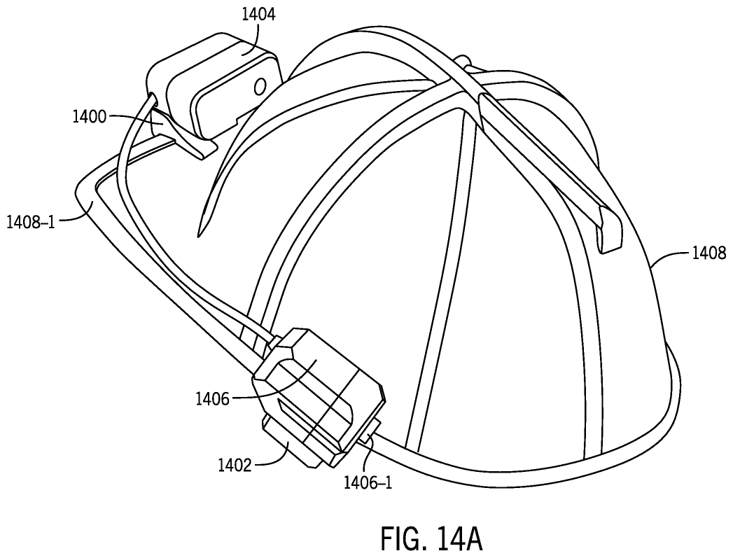

FIGS. 13A-13C depict various views of another example mounting accessory 1300 and module 1302. Specifically, FIG. 13A depicts a front sectional view of mounting accessory 1300 that is used for mounting module 1302 to a user's/wearer's mask 1304. Mounting accessory 1300 is attached directly to bezel 1304-1 of mask 1304 as shown in FIG. 13A. FIG. 13B depicts an exploded view of module 1302, mounting accessory 1300 and mask 1304. Mounting accessory 1300 is directly to module 1302 in this embodiment as best illustrated in FIG. 13C. Mounting accessory 1300 employs dual plates 1300-1, 1300-2 (structure) separated by front and rear spacers 1300-3,1300-4 that together function as a clamp or clamp mechanism. Plates 1300-2 that is closest to module 1302 comprises two hooks or claws 1300-2a and 1300-2b and plate 1300-1 comprises two corresponding panel portions 1300-1a, 1300-1b that together define an angular open channel to receive and frictionally fit brim 1304-1 around the transparent enclosure (mask). Mounting accessory 1300 is positioned to receive brim 1304-1 at an angle and bent to snap mounting accessory 1300 into place onto brim 1304-1.

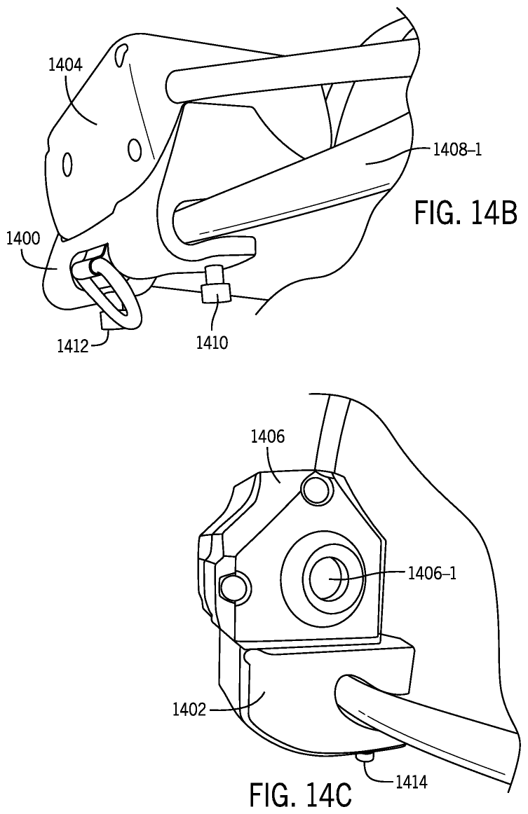

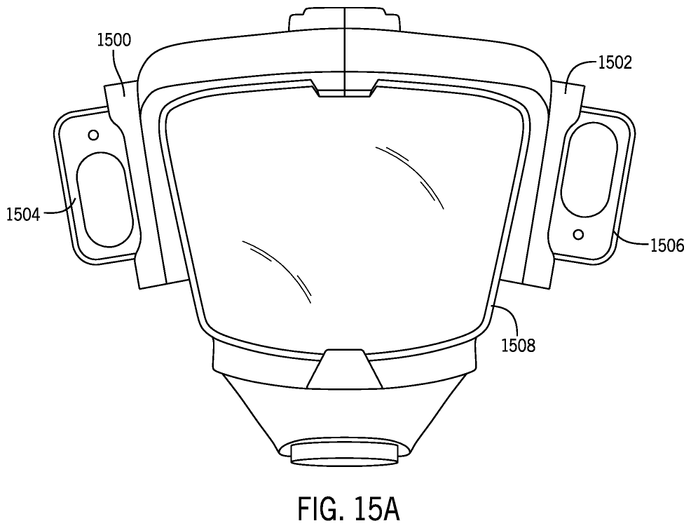

FIGS. 14A-14C depict another embodiment of mounting accessories 1400, 1402 that mounts dual modules 1404, 1406 to a firefighter's helmet 1408. FIG. 14A depicts a front perspective view of dual modules mounted 1404, 1406 mounted to helmet 1408 along its brim 1408-1. FIG. 14B depicts a sectional view of the rear of helmet 1408 (illustrating a module) and FIG. 14C depicts a sectional view of the side of the helmet (illustrating a module). In this embodiment, modules 1404, 1406 are connected by an electrical cord and are snapped into mounting accessories 1400, 1402 and clamped directly to brim 1408-1 of helmet 1408. Mounting accessories 1400 have two screw clamps 1410, 1412 as shown in FIG. 14B to clamp or secure module 1400 to brim 1408. Mounting accessory 1402 also has two screws 1414 (second set not shown) to clamp or secure modules 1406 to brim 1408-1 as shown in FIG. 14C. These modules then sit on brim 1408-1 by friction. Module 1402 in the rear of helmet 1404 includes a battery, MCU, IMU, GPS and environmental sensors as described herein above and module 1406 clamped along the side of helmet 1408 includes an IR camera 1406-1 as described hereinabove. Most of the weight of the modules are now in the rear of the helmet.

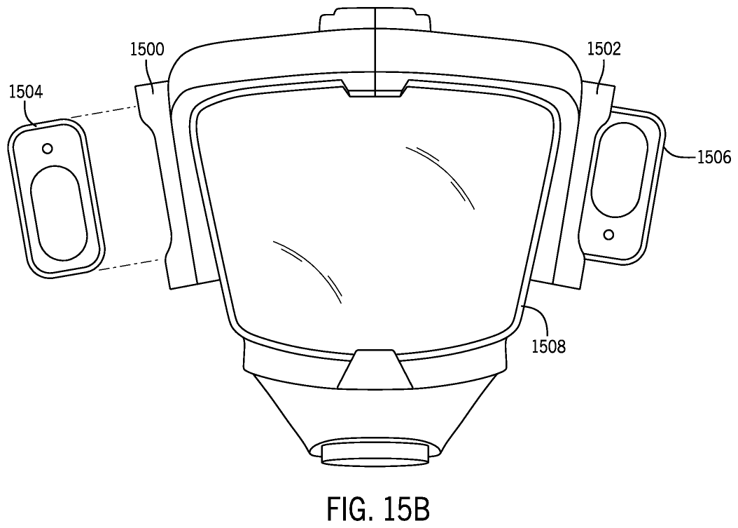

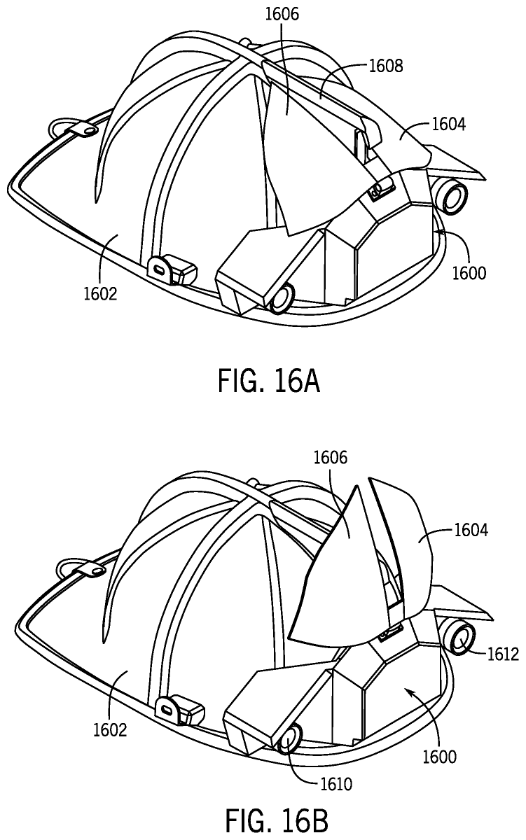

FIGS. 15A-15B depict front views of another embodiment of mounting accessories 1500, 1502 for mounting modules 1504, 1506 to SCBA mask 1508. Mounting accessories 1400, 1402 are friction fit (brackets) onto mask 1508 along the sides thereof adjacent ear area in a clamping motion similar to other embodiments herein, but could alternatively be made as an integral unit with mask 1508. Modules 1504, 1506 are held securely in place by magnetic elements positioned in the bay or receiving area (conforming to the shape of side of modules) of mounting accessories 1500, 1502 as well as the modules 1504, 1506 themselves.

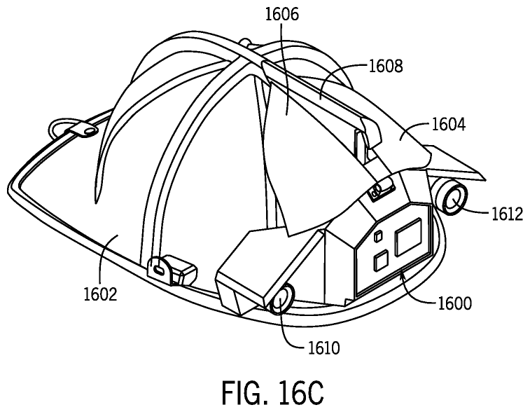

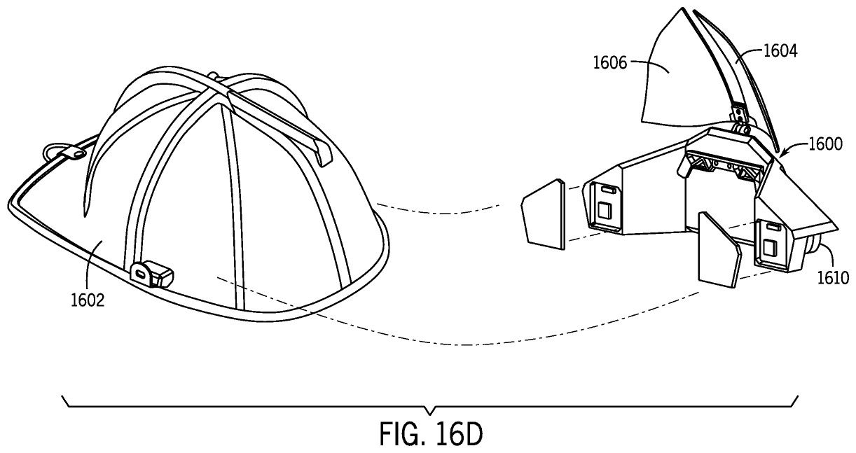

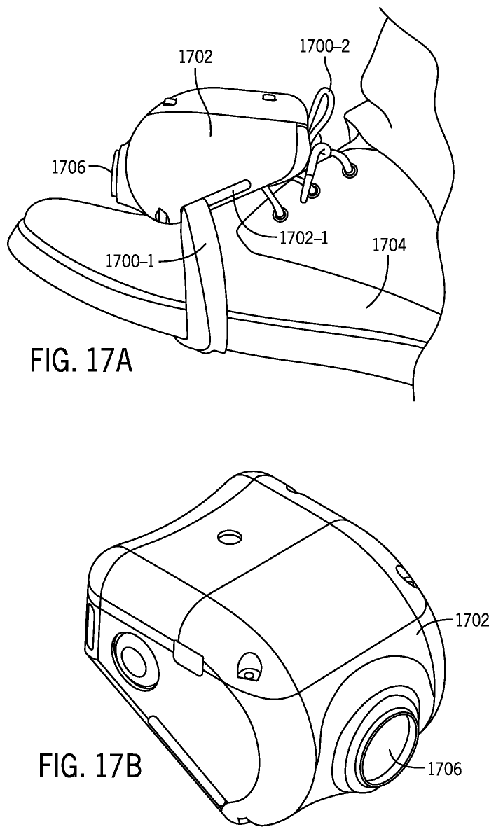

FIGS. 16A-16D depict various views of another embodiment of mounting accessory that functions to clamp a module to a helmet. In this embodiment, mounting accessory and module, together 1600 are configured as a single integral component. Mounting accessory/module 1600 is configured as a shield mount wherein accessory/module 1600 will mount to the front of the brim of helmet 1602. Screws are used herein to clamp accessory 1600 to opposing points on the brim of helmet 1602, but to avoid interfering with a firefighter's (wearer or user) helmet crest with identification information. The screws are in the back of accessory 1600 which mounts to existing shield holes as known to those skilled in the art. Flaps or baffles 1604, 1606 are configured to pivot downward over the top of helmet 1602 to engage opposing parts of helmet 1602 separated by ridge 1608 of helmet 1602. Baffles 1604, 1606 are used for stability as well as heat deflection. Module incorporates several components including a battery, MCU, IMU, GPS and environmental sensors as described hereinabove. FIG. 16C shows an open enclosure of the accessory module. A single IC board is depicted as the computer unit (e.g., computer Raspberry Pi single board computer) with many of these components (e.g., MCU, IMU and GPS) and behind it is the battery. Accessory/module 1600 incorporate two opposing IR cameras 1610, 1612 that are connected to the MCU. IR Cameras 1610, 1612 are used to look at the space to determine depth for localization. Data points are obtained and sent to mobile device 108 for comparison and analysis.

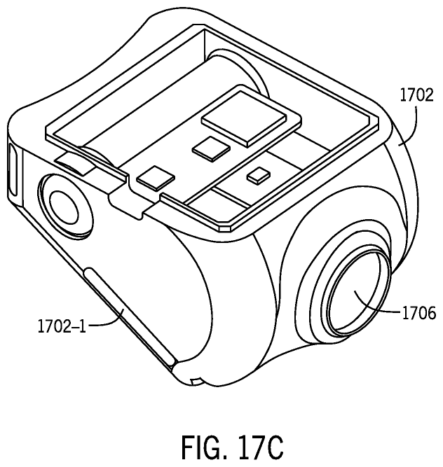

FIGS. 17A-17C depict various views of another example mounting accessory (as 1700-1 and 1700-2, described below) and/or module 1702. Specifically, FIG. 17A depicts a side view of module 1702 mounted to a user's foot. FIG. 17B depicts a perspective view of module 1702. FIG. 17C depicts a perspective view of module 1702 with the housing or enclosure open exposing an IC board. Mounting accessory includes (1) elastic strap 1700-1 that extends through a slot 1702-1 in the housing of module 1702 and a strand on the end of module 1702. Strap 1700-1 mounts around the shoe 1704 of the wearer or user while the strand 1700-2 secures the end of module housing to the shoelaces of the wearer's shoe 1704. Strap 1700-1 is preferably elastic to fit snugly around shoe 1704. Module 1702 may include components described above, including battery, MCU, GPS and/or IMU (as examples) or any desired components. Module 1702 is configured to communicate with other modules on PPE or the user itself as described above, or alternative directly to mobile device 108. In this embodiment, module 1702 includes a flashlight to enhance user visibility.

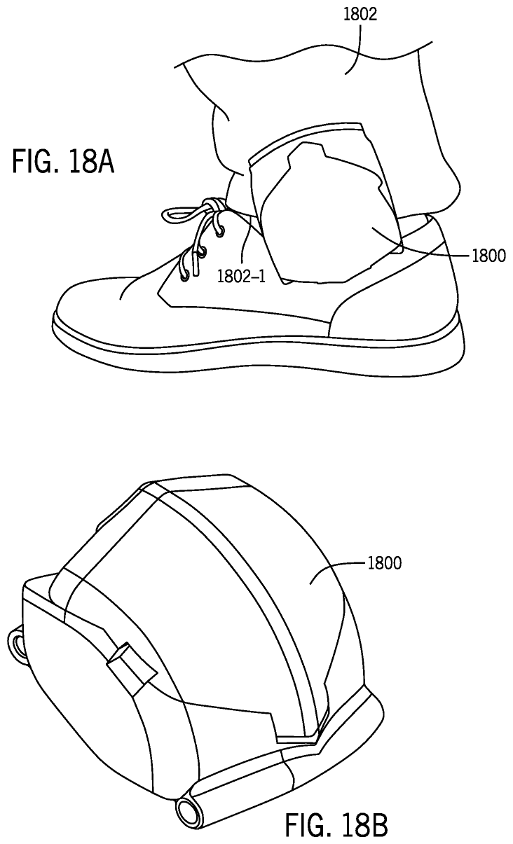

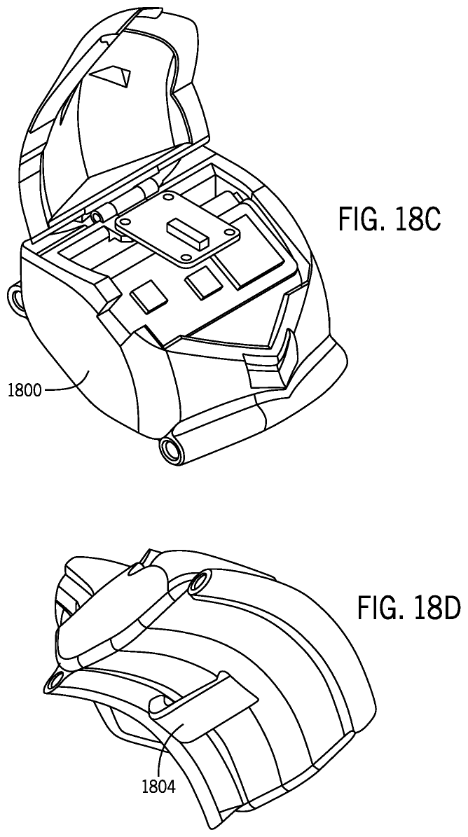

FIGS. 18A-18D depict various views of an ankle mounting module 1800. Specifically, FIG. 18A depicts a side rear view of module 1800 mounted to a wearer or user 1802 along his/her ankle 1802-1 using clip 1804 as a mounting accessory. In this embodiment, module 1800 has some of the components described above including battery, MCU, GPS and/or IMU. However, in this embodiment, there is no flashlight. FIG. 18B depicts a rear perspective view of module 1800. FIG. 18C depicts a front perspective view of module 1800 with its door open exposing internal components. FIG. 18D depicts a side view of clip 1804 attached to the inside of module 1800.

In summary with respect to mounting accessories described above, one or more example embodiments are disclosed module attachments intended to retrofit existing protective equipment. In some example configurations, some or all sensor integration is designed as an add-on to existing respirator visors, facemasks, helmets, or gloves. For example, such add-ons may include multiple sensors, cameras, optics, lighting, and/or communication subsystems that are integrated into an enclosure module. These units are either attached to the equipment or worn around the user's neck, placed in a boot as examples. In example embodiments, the modules of the apparatus described herein are modular, removable, and/or ruggedized. The modules are preferably utilized in conjunction with an accessory mount, which clamps, bolts, or otherwise attaches onto the protective equipment. In accordance with embodiments herein, these clamp systems do not fundamentally change the function(s), seal, protective nature, gasket, weatherproofing, ballistic ability, or respiration functionality of the respirator. The module(s) is mechanically clamped with the use of fasteners and mounted to the existing shape, bezel, or general form-factor of the original equipment, according to example embodiments. In some embodiments, existing accessory mounting points may also be used.

In one embodiment, a t-rail system performs the same function. In yet another embodiment, one or more magnets secure the modules in place and assist in providing haptic feedback indicative of a secure connection. The modules can be replaced or swapped based on the needs of the operator. Thus, the mounting accessory and module allow for swap-ability between sensors and integration of various cameras, microphones, sensors, batteries, microcontrollers, displays, sensors, optics, microphones, and/or other peripherals into the platform.

Each module allows for both power and data to be transferred between the microcontroller module and the individual sensor components. The resulting system provides both power and data, which may utilize an I2C system bus, for example. The information taken by the sensors is integrated into a scalable infrastructure that transmits data to a backend server. This keeps the platform updated with improvements made to sensor technology, as some or all modules can be added or removed via an interchangeable sensor architecture, according to some examples.

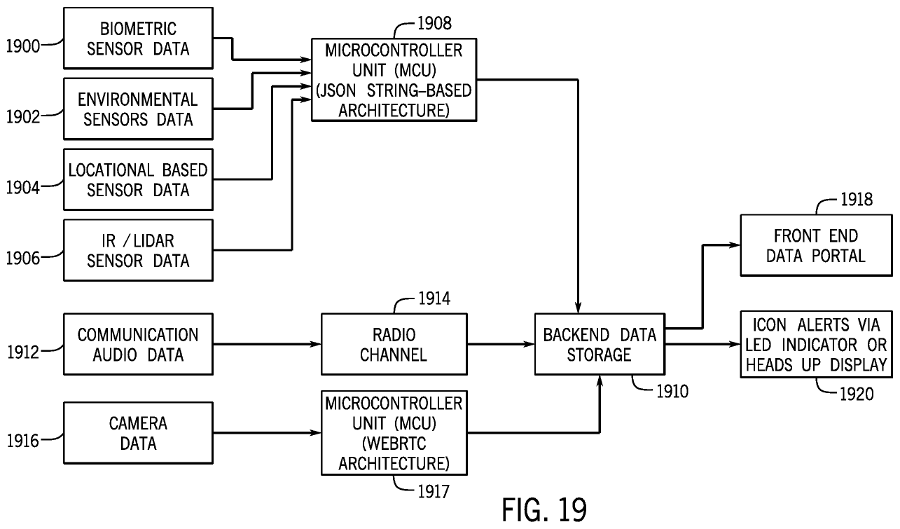

FIG. 19 depicts a block diagram of data flow of the system shown in FIG. 2. In particular, biometric sensor data 1900, environmental sensors data 1902, locational based sensor data (IMU) 1904, IR camera and Lidar sensor data 1906 is transmitted to microcontroller (MCU) 1908 and then sent on to backend data storage 1910 (part of remote central computer system 112, for example). Communication audio data 1912 is transmitted via radio channel 1914 and then on to backend data storage 1910. Camera data 1916 is transmitted to another microcontroller unit (MCU) 1917 and then transmitted to backend data storage 1910. The back end data stored in the backend data storage is used for a front end data portal 1918 as well as icon alerts via LED indicators or heads up display 1920.

### Interactive Data Portal

The user interface described above, usable on mobile device 108 as shown in FIG. 2 or any computer via a web application, allows for the display of video data, biometric, locational, environmental, or other sensor data. All sensors are preferably integrated into a rugged and scalable infrastructure. The specific sensor agnostic approach allows for modularity in sensor selection and connectivity options. This data portal has graphical user interface screens adapted to each user's preferences. Additionally, industry-specific embodiments and interfaces that feature specific visualizations such as mapping, task lists, camera feeds, and customizable interfaces, can be provided.

An alternate embodiment can run on the Android, Apple, Google, or Microsoft respective operating systems, such as by downloading from one or more respective application (app) stores. An embodiment of this database may be built on Amazon Web Services, for example. Another embodiment is built utilizing Grafa components, while another embodiment is built on Bubble.io, and another is built on MIT App Inventor 2, which feature alternate placements, emphasizing to reflect needs of various industries.

### Interactive Data Portal Data Management

Data is transferred over a low-energy Bluetooth. Alternatively, the data transmissions described herein can also incorporate WIFI, phone network, LoRa, UWB, or Iridium, for example. A JSON architecture can be utilized to allow for flexibility in component selection and connection to existing software packages. A WebRTC architecture can be utilized for streaming audio and visual data. In one embodiment, the biometric, environmental, and locational data is passed over JSON while visual is over WebRTC and audio communication is over radio, but other methodologies to pass this data alternatively or additionally may be used. The controller portal has intuitive and simple displays to monitor the wellbeing and whereabouts of multiple operators. This provides operational control over what is displayed on the portal and heads up display.

### Interactive Data Portal Icon Based Monitoring System

The sensors module's infrastructure allows for swappable components and is built in a modular way that allows for custom sensor packages for unique requirements. For example, an icon-based heads-up display to allow the display of more dynamic text-based instructions, shared maps, and compasses. As mentioned previously, threshold alerts can be altered based on preset preference(s) loaded into the application. Individuals can also be added to the data portal through custom loading profiles entered manually, or scanned via a CaC card, RFID, or other contact based systems, for example.

An attendant/commanding officer or other user can program actionable LED alerts driven by actionable commands. Commands are then driven by changes to color and blinking frequency by the LEDs in the Heads-up-Displays (HuD). Additionally, a smart onboard assistant can be integrated that can take voice commands and custom tailor the HuD to each user's unique preferences.

In some embodiments, these alerts are color coded based on severity. For example, if temperature or hazardous elements near an operator are below a threshold they are green. If at dangerous threshold, they are yellow. If at a very dangerous threshold, they turn red.

### Interactive Data Portal Optical Camera Feed

Potential optical streams could include infrared, night vision, as well as simple optical cameras. These vision modes can range from low-count pixel arrays to full 4 k displays (or others). These allow both live streaming as well as a review after operations. Additionally, the user can tag, highlight, and rewind video through the application.

### Interactive Data Portal Customizable Task List

A user, such as an attendant/commanding officer can compile, store, and/or display a personal or group task list for display on one or more Heads-up-Displays or other display screens. Additionally, users can drag and drop operators to assign them to tasks as well as drag those tasks to portions of the map for assignment orders.

### Interactive Data Portal Mapping and GPS Interface

Through simultaneous localization and mapping (SLAM), 3D maps of a particular structure or space can be generated and displayed as either a 3-dimensional render of the given space or a 2-dimensional floor plan. The geospatial mapping information needed to generate either asset is collected through a selected combination of stereo video data utilizing both visible spectrum and infrared spectrum cameras, a LiDAR (distance), ultrasonic (distance), inertial measurement unit (acceleration, magnetometer, gyroscope data in the X, Y, and Z axis), Ultra-Wideband (UWB) time-of-flight localization, and GNSS data (latitude, longitude), according to a preferred embodiment.

Stereo video streams may be used to recreate the 3D high density LiDAR point clouds used in traditional simultaneous localization and mapping (SLAM) embodiments. A convolutional neural network may be used to predict the positional measurement of each shared pixel on the current video frame. The network is trained using a dataset composed of stereo video and correlated point clouds of environments meant to simulate fire emergencies (useDEFOG). These predicted pixel measurements are projected into a point cloud bounded by the focal cone of the cameras.

GPS data is used to help identify the global frame location of the operator in the structure. The LiDAR and ultrasonic data are used to generate various distances between walls, floors, ceilings and the operator for the purpose of graph depth correction of estimated point clouds. As the operator moves throughout the structure, the point of reference of the sensors changes. To track these changes, accelerometer and/or gyroscopic data can be used to track the point of reference of the operator, and thus, the relative location of the sensor suite.

If multiple operators are present on scene the mapping task can be optimized through collaborative SLAM techniques. The embodiment of each operator is responsible for a local map of the environment and its path. When the data of all embodiments are combined, the global map constructed is more resilient to sensor inaccuracies, experiences faster loop closure (precise alignment of the global map), and near true relative localization between operators.

The geospatial mapping may be used to track the position and movement of operators throughout the structure. This tracking may also include tracking as operators ascend and descend staircases, ramps, or ladders, and can also be used to identify sudden falls, such as when the Z axis of the accelerometer indicates prolonged acceleration against the gravity access. This information can also be logged locally or stored virtually for later review, such as during training exercises, investigations, or general documentation.

It is to be understood that this disclosure teaches examples of the illustrative embodiments and that many variations of the invention can easily be devised by those skilled in the art after reading this disclosure and that the scope of the present invention is to be determined by the claims below.

What is claimed is:

1. An apparatus for mounting on personal protective equipment (PPE) of a user located on premises in a hazardous environment, the apparatus configured for providing data monitoring and communication of the user for remote review, analyses and/or user deployment and navigation guidance in the hazardous environments to enhance incident command capability, the personal protective equipment including a helmet and/or a mask, the apparatus comprising:
(a) one or more modules including:
an inertial measurement unit for measuring and reporting acceleration, velocity and position data of the user on premises;
an infrared camera for creating image data as the user moves through the premises;
a GPS receiver for generating geolocation data of the user via satellite imagery as the user enters the premises;
an ultrasound sensor for generating data relating to the distance between the user and objects on premises; and
a microcontroller unit for processing and transmitting data from the inertial measurement unit, infrared camera, ultrasound sensor and GPS to a remote computer system; and
(b) a mounting accessory for mounting the one or more modules to the user's personal protective equipment.

2. The apparatus of claim 1 wherein the one or more modules further includes one or more environmental sensors including a barometric pressure sensor for calculating relative altitude data of the user, a toxicity sensor for sensing the toxicity of a compound that is dangerous to the user, a temperature sensor for sensing the temperature of the environment and/or a pressure sensor for sensing the pressure in an air tank of the PPE.

3. The apparatus of claim 1 wherein the one or more modules further includes a biometric sensor for measuring health information of a user including body temperature, heart rate, and blood oxygen.

4. The apparatus of claim 1 wherein the mounting accessory is configured to clamp to a brim of the helmet or a bezel of the mask.

5. The apparatus of claim 1 wherein the one or more modules and mounting accessory are integrated as one piece.

6. The apparatus of claim 1 wherein the mounting accessory includes one or more magnets to secure the one or modules to the personal protective equipment on the user.

7. A system comprising:
(a) apparatus for mounting on personal protective equipment (PPE) of a user located on premises in a hazardous environment, the apparatus configured for providing data monitoring and communication of the user for remote review, analyses and/or user deployment and navigation guidance in the hazardous environments to enhance incident command capability, the personal protective equipment including a helmet and/or a mask, the apparatus including a user tracking device for tracking location of the user on premises comprising:
an inertial measurement unit for measuring and reporting acceleration, velocity and position data of the user on premises;
an infrared camera for creating image data as the user moves through the premises;
a GPS receiver for generating geolocation data of the user via satellite imagery as the user enters the premises;
an ultrasound sensor for generating data relating to the distance between the user and objects on premises; and
a microcontroller unit for processing and transmitting data from the inertial measurement unit, ultrasound sensor and GPS remotely; and
(b) a mounting accessory for mounting the user tracking device to the user's personal protective equipment.

8. The system of claim 7 further comprising (c) a computer system in communication with the microcontroller unit via a network for determining and/or generating indoor configuration of the premises and a location and a direction of the user on the premises based on the data received from the microcontroller unit.

9. The apparatus of claim 7 wherein the user tracking device further comprises an infrared camera for creating image data as the user moves through the premises.

10. The apparatus of claim 7 further including one or more environmental sensors including a barometric pressure sensor for calculating relative altitude data of the user, a toxicity sensor for sensing the toxicity of a compound that is dangerous to the user, a temperature sensor for sensing the temperature of the environment and/or a pressure sensor for sensing the pressure in an air tank of the PPE.

11. The system of claim 7 further including a biometric sensor for measuring health information of a user including body temperature, heart rate and/or blood oxygen.

12. The system of claim 7 wherein the mounting accessory is configured to clamp to a brim of the helmet or a bezel of the mask.

13. The system of claim 7 wherein the mounting accessory includes one or more magnets to secure the one or modules to the personal protective equipment on the user.

14. An apparatus that is configured as one or more modules or components to be mounted on a user on premises in hazardous environments, the apparatus comprising:
(a) a first user tracking device for tracking location of a user on the premise, the first user tracking device including:
an inertial measurement unit for measuring and reporting acceleration, velocity and position data of the user on premises;
a GPS receiver for generating geolocation data of the user via satellite imagery as the user enters the premises; and
a microcontroller unit for processing and transmitting data from the inertial measurement unit and GPS to a remote computer system; and
(b) a second user tracking device including:
an inertial measurement unit for measuring and reporting acceleration, velocity and position data of the user on premises;
a GPS receiver for generating geolocation data of the user via satellite imagery as the user enters the premises; and
a microcontroller unit for processing and transmitting data from the inertial measurement unit and GPS to the microcontroller the remote computer system,
wherein the first tracking device and second tracking device are configured to transmit data therebetween; and
(c) a first mounting accessory and second mounting accessory for mounting the first user tracking device and the second user tracking device respectively to the user.

15. The apparatus of claim 14 wherein the first tracking device includes an ultrasound sensor for generating data relating to the distance between the user and objects on premises.

16. The apparatus of claim 14 further comprising a computer system for determining and/or generating indoor configuration of the premises and a location and a direction of the user on the premises based on data from the user tracking device and ultrasound sensor.

17. The apparatus of claim 14 wherein the first tracking device includes an infrared camera for creating image data as the user moves through the premises.

18. The apparatus of claim 14 further comprising one or more environmental sensors including a barometric pressure sensor for calculating relative altitude data of the user, a toxicity sensor for sensing the toxicity of a compound that is dangerous to the user, a temperature sensor for sensing the temperature of the environment and/or a pressure sensor for sensing the pressure in an air tank of the PPE.

19. The apparatus of claim 14 further comprising one or more biometric sensors for measuring health information of a user including body temperature, heart rate and/or blood oxygen.
